(19) United States
(12) Patent Application Publication
(10) Pub. No.: US 2025/0271427 A1
(43) Pub. Date: Aug. 28, 2025

(54) RICKETTSIA IGM ASSAY

(71) Applicant: VMRD, INC., Pullman, WA (US)

(72) Inventor: Lee Fuller, Fullerton, CA (US)

(21) Appl. No.: 18/877,134

(22) PCT Filed: Jun. 23, 2023
(86) PCT No.: PCT/US2023/068984

§ 371 Date: Dec. 19, 2024

Related U.S. Application Data
(60) Provisional application No. 63/354,798, filed on Jun. 23, 2022.

(51) Int. Cl. G01N 33/569; C07K 14/29

(52) U.S. Cl. CPC G01N 33/56911; C07K 14/29; G01N 2333/29; G01N 2469/20

(57) ABSTRACT

Disclosed herein is use of a Rickettsia OmpB β-peptide epitope that binds host immune IgM as a specific diagnostic substrate for detecting patient immune IgM antibody to both spotted fever group and typhus group Rickettsia.

### CROSS-REFERENCE TO RELATED APPLICATION

This application claims the benefit of, and priority to, U.S. Provisional Patent Application 63/354,798, filed Jun. 23, 2022, the entire contents of which is incorporated by reference herein.

### SEQUENCE LISTING

A Sequence Listing is submitted herewith and incorporated by reference herein as an XML file created on Jun. 21, 2023, entitled “1960082-00006_Sequence_Listing.xml” and having a size of 6 KB.

### BACKGROUND

The genus Rickettsia is a collection of obligate intracellular Gram-negative bacteria found in ticks, lice, fleas, mites, chiggers, and mammals. These zoonotic pathogens cause infections that disseminate in the blood to many organs. Most rickettsial pathogens are transmitted directly to humans by infected arthropod vectors (i.e., fleas, lice, mites, or ticks) during feeding. Rickettsia also might be transmitted when a person inadvertently inoculates the arthropod bite wound (or other breaks in the skin) with rickettsial pathogens; this can happen by scratching skin contaminated with an arthropod's infectious fluids or feces, or by crushing the arthropod vector at the bite site. Inhaling bacteria or inoculating conjunctiva with infectious material also can initiate infection for some rickettsial pathogens.

Many clinical laboratories both domestic and internationally continue to use whole-cell indirect immunofluorescence assay (IFA) and micro-immunofluorescence (MIF) assays for Rickettsia serology. Indeed, IFA is still considered the “Gold Standard” assay.

However, the chief drawback of whole-cell antigen approach is the lipopolysaccharide (LPS) antigen which is the main “false-target,” producing false-positives due to cross reactivity of IgM with enterobacterial LPS. This natural immunity is sufficiently strong as to preclude serum absorption techniques, and LPS cannot be selectively removed from the crystalline s-layer of the intact whole cell Rickettsia antigen.

Thus, improved methods would be beneficial.

### SUMMARY

The instant disclosure provides improved epitopes, polypeptides, peptides, systems, devices, and methods for detection of Rickettsia infection. For example, disclosed methods provide an Enzyme-Linked Immunosorbent Assay (ELISA) for the selective detection of rickettsia in mammalian serum.

Disclosed polypeptides and peptides comprise epitopes comprising a Rickettsia outer membrane protein B (OmpB) epitope, specifically the β-peptide of the epitope, that binds host immune IgM as a specific diagnostic target or substrate for detecting patient IgM antibody to spotted fever and typhus group Rickettsia.

Disclosed systems and devices comprise polypeptides and peptides comprising a Rickettsia outer membrane protein B (OmpB) epitope, specifically the β-peptide of the epitope, that binds host immune IgM as a specific diagnostic target or substrate for detecting patient IgM antibody to spotted fever and typhus group Rickettsia, as well as hardware and equipment suitable for use in immunoassays.

Disclosed methods comprise use of a polypeptide or peptide comprising Rickettsia outer membrane protein B (OmpB) epitope, specifically the β-peptide of the epitope, that binds host immune IgM as a specific diagnostic target or substrate for detecting patient IgM antibody to spotted fever and typhus group Rickettsia.

Disclosed methods reduce the false-positive rate long associated with traditional methods of testing for Rickettsia infection.

Disclosed methods reduce the cost of determining the presence of Rickettsia infection.

Disclosed methods further comprise treatment of a Rickettsia infection in the case of a positive result to a Rickettsia infection assay, or further diagnostic testing in the case of a negative result to a Rickettsia infection assay.

### BRIEF DESCRIPTION OF THE FIGURE

FIG. 1 shows a Western blot produced using two antigens that had been blocked by OmpA and one (R. conorii) that had not been blocked in the described 18-antigen assay. On the left side the blot was treated overnight with trypsin and the ˜25 kDa epitope is seen in all three examples.

### DETAILED DESCRIPTION

Disclosed herein is use of a polypeptide comprising a Rickettsia OmpB epitope that binds host immune IgM as a specific diagnostic substrate for detecting patient IgM antibody to spotted fever group Rickettsia.

The antibody response to Rickettsia infection is primarily directed against group-common (spotted fever [SFG] or typhus [TG] groups) lipopolysaccharide (LPS) and 1-2 outer-membrane attachment proteins (OmpA and/or OmpB). Standard IFA and MIF methods detect antibodies against the whole organism and remain the most commonly used diagnostic methods, though not necessarily the most accurate; IgM-specific antibody assays are particularly prone to false-positive reactivity with whole cell antigens due to the LPS antigen.

### Disclosed Polypeptides/Peptides/Epitopes

In contrast to current assays, the instant disclosure provides polypeptides and peptides comprising a Rickettsia outer membrane protein B (OmpB) epitope, comprising the β-peptide, that binds host immune IgM as a specific diagnostic target or substrate for detecting patient IgM antibody to spotted fever and typhus group Rickettsia.
SEQ ID NO:1 shows the DNA sequence used in development of the polypeptides and peptides comprising the active recombinant form of β-peptide.
SEQ ID NO:2 shows the amino acid sequence used in development of the polypeptides and peptides comprising the active recombinant form of β-peptide.
SEQ ID NO:3 shows the amino acid sequence used in development of the polypeptides and peptides comprising the active recombinant form of β-peptide with “tags.”

Disclosed polypeptides and peptides display an increased specificity for both spotted fever and typhus group organisms. This specificity eliminates the need for an initial (and expensive) step of removing competing IgG class antibody to produce a sensitive assay.

Disclosed polypeptides can comprise 10%, 20%, 30%, 40%, 50%, 60%, 70%, 80%, 90%, or 100% sequence identity with SEQ ID NO:2.

Disclosed peptides can comprise 10%, 20%, 30%, 40%, 50%, 60%, 70%, 80%, 90%, or 100% sequence identity with SEQ ID NO:2.

Disclosed polypeptides can comprise functional analogs comprising 10%, 20%, 30%, 40%, 50%, 60%, 70%, 80%, 90%, or 100% sequence identity with SEQ ID NO: 2.

Disclosed peptides can comprise functional analogs comprising 10%, 20%, 30%, 40%, 50%, 60%, 70%, 80%, 90%, or 100% sequence identity with SEQ ID NO:2.

Disclosed polypeptides and peptides can be recombinant.

Disclosed polypeptides and peptides can be naturally-occurring.

Disclosed polypeptides and peptides can be non naturally occurring.

### Disclosed Systems and Devices

Disclosed systems and devices comprise a polypeptide or peptide comprising a Rickettsia outer membrane protein B (OmpB) epitope, specifically the β-peptide, or functional analogs thereof, that bind host immune IgM as a specific diagnostic target or substrate for detecting patient IgM antibody to spotted fever and typhus group Rickettsia.

In further embodiments, disclosed systems comprise devices and equipment for performing immunoassays such as reaction “plates” including 96-well plates. In embodiments, the reaction plate wells comprise a Rickettsia antigen, for example an OmpB epitope, specifically the β-peptide. In embodiments, a Rickettsia outer membrane protein B (OmpB) β-peptide epitope is added to a well and incubated overnight, then directly back-coated to preserve antigenicity, then emptied and dried. Further embodiments can comprise packaging the reaction plate.

Disclosed systems can further comprise plate “readers” to quantify reaction results. For example, disclosed systems can comprise an enzyme immunoassay (EIA) reader set to “read” at, for example 450 nm.

Disclosed systems can comprise buffers such as, for example, PBS, TWEEN-20, and the like.

### Disclosed Methods of Use

Disclosed methods comprise use of a polypeptide or peptide comprising a Rickettsia outer membrane protein B (OmpB) epitope, specifically the β-peptide, or functional analogs thereof, that bind host immune IgM as a specific diagnostic target or substrate for detecting patient IgM antibody to spotted fever and typhus group Rickettsia.

For example, in disclosed embodiments, methods of use can comprise performing an immunoassay to determine the presence of Rickettsia infection. For example, disclosed methods can comprise identification of a patient suspected of having a Rickettsia infection. Sera collected from the identified patient can be added to a well in a reaction plate prepared as described above, said well containing the B (OmpB) epitope, specifically the β-peptide, and to the individual rows at 1:100 in PBS containing Tween-20 (standard EIA wash buffer) and incubated for 60 minutes at ambient temperature (˜23 C). Wells are then washed 3× with this Wash Buffer, tapped to remove buffer before adding 100 uL peroxidase-conjugated anti-human IgG for 30 minutes (same temperature).

In embodiments, after washing again, 3,3′,5,5″-tetramethylbenzidine (TMB) substrate is added for a timed period such as 10 minutes, and the reaction is stopped by addition of 100 uL of 10% NaOH. An EIA reader set to, for example, 450 nm is used to read the absorbance for each well.

Further embodiments comprise a “cut-off” serum with a 450 nm absorbance value squarely between the highest positive and lowest negative values. High values than this cut-off are considered positive and lower values denote negative.

Disclosed methods reduce the false-positive rate long associated with traditional methods of testing for Rickettsia infection. For example, disclosed methods avoid producing false-positives due to cross reactivity of IgM with enterobacterial LPS

Disclosed methods reduce the cost of determining the presence of Rickettsia infection. For example, disclosed epitope polypeptides and peptides display an increased specificity for both spotted fever and typhus group organisms. This specificity eliminates the need for an initial (and expensive in terms of time and cost) step of removing competing IgG class antibody to produce a sensitive assay.

Disclosed methods further comprise treatment of a Rickettsia infection in the case of a positive result to a Rickettsia infection assay. For example, in the case of a positive result to a Rickettsia infection assay, antibiotic therapy initiated early in the first week of illness is highly effective and is associated with the best outcome. Fever usually subsides within 24-72 hours after starting antibiotic therapy.

In embodiments, treatment can be terminated 2-3 days after the patient is afebrile and at least 10 days of therapy has been given. In embodiments comprising antibiotic administration, doxycycline can be administered; it is preferred over other tetracyclines for treatment of Rickettsial infections and, at such low dose and short duration, is rarely associated with staining of teeth in children younger than 8 years. Chloramphenicol may be used as an alternative. However, it is rarely used in the United States because of its potential bone marrow toxicity. Recent data from Europe suggest that fluoroquinolones, such as ciprofloxacin and ofloxacin, may be effective in the treatment of certain rickettsioses.

Disclosed methods can comprise further testing and/or evaluation in the case of a negative result to a Rickettsia infection assay. For example, in the case of a negative result to a Rickettsia infection assay, additional diagnostic testing can be performed to identify the cause of the patient's symptoms.

### Disclosed Kits

Disclosed kits comprise devices useful in performing the disclosed methods. For example, disclosed kits can comprise a polypeptide or peptide comprising a Rickettsia outer membrane protein B (OmpB) epitope, specifically the β-peptide or functional analogs thereof, that bind host immune IgM as a specific diagnostic target or substrate for detecting patient IgM antibody to spotted fever and typhus group Rickettsia.

In further embodiments, disclosed kits can comprise devices and equipment for performing immunoassays such as “reaction plates” such as 24-, 48-, or 96-well plates. In embodiments, the reaction plate wells comprise a Rickettsia antigen, for example a polypeptide or peptide comprising an OmpB epitope, specifically the β-peptide, or a functional analog thereof.

In embodiments, a polypeptide or peptide comprising a Rickettsia outer membrane protein B (OmpB) β-peptide epitope is added to a well and incubated overnight, then directly back-coated to preserve antigenicity, then emptied and dried. Further embodiments can comprise packaging the reaction plate.

Disclosed kits can further comprise instructions for performing the methods disclosed herein.

Use of disclosed kits can reduce the false-positive rate long associated with traditional methods of testing for Rickettsia infection. For example, disclosed methods avoid producing false-positives due to cross reactivity of IgM with enterobacterial LPS

Use of disclosed kits can reduce the cost of determining the presence of Rickettsia infection. For example, disclosed epitopes display an increased specificity for both spotted fever and typhus group organisms. This specificity eliminates the need for an initial (and expensive in terms of time and cost) step of removing competing IgG class antibody to produce a sensitive assay.

### Example 1—Antigen Comparison

Table 1 depicts reactivity of patient sera against 18 antigens in IgM ELISA. A panel of 18 spotted fever group (SFG) species were assembled which express the OmpB epitope, plus R. felis (transition species) and SFG LPS as a control.

These antigens were purified from cell culture with LPS removed by two sequential methods. These antigens consist of OmpA and OmpB heteroduplex isolated from Rickettsial (SFG) s-layer protein:

| 1 | 2 | 3 | 4 | 5 | 6 | 7 | 8 | 9 | 10 | 11 | 12 |
|---|---|---|---|---|---|---|---|---|---|---|---|
| 0.136 | 0.124 | 0.094 | 0.262 | 0.281 | 0.242 | 0.293 | 0.199 | 0.054 | 0.108 | 0.054 | 0.282 |
| 0.192 | 0.136 | 0.115 | 0.719 | 0.624 | 0.751 | 0.699 | 0.346 | 0.072 | 0.107 | 0.061 | 0.576 |
| 0.091 | 0.170 | 0.070 | 0.251 | 0.208 | 0.203 | 0.251 | 0.203 | 0.060 | 0.073 | 0.063 | 0.233 |
| 0.261 | 0.157 | 0.193 | 0.834 | 0.895 | 0.997 | 0.854 | 0.516 | 0.097 | 0.174 | 0.084 | 0.701 |
| 0.129 | 0.120 | 0.096 | 0.486 | 0.406 | 0.482 | 0.442 | 0.233 | 0.067 | 0.100 | 0.104 | 0.411 |
| 0.071 | 0.104 | 0.066 | 0.115 | 0.130 | 0.104 | 0.127 | 0.091 | 0.048 | 0.067 | 0.049 | 0.116 |
| 0.225 | 0.164 | 0.153 | 0.483 | 0.465 | 0.489 | 0.483 | 0.310 | 0.114 | 0.189 | 0.119 | 0.447 |
| 0.218 | 0.156 | 0.105 | 0.297 | 0.324 | 0.252 | 0.3.3 | 0.195 | 0.065 | 0.111 | 0.107 | 0.277 |
| Afric | Parke | Sib | Conor | Slow | Hon | Rick | Lao | Hbil | Mont | Raoul | ambi |

| 13 | 14 | 15 | 16 | 17 | 18 | 19 | 20 |
|---|---|---|---|---|---|---|---|
| 0.232 | 0.409 | 0.079 | 0.336 | 0.057 | 0.051 | 0.110 | 0.097 |
| 0.344 | 1.049 | 0.079 | 0..936 | 0.063 | 0.052 | 0.114 | 0.089 |
| 0.149 | 0.395 | 0.079 | 0.309 | 0.071 | 0.063 | 0.107 | 0.077 |
| 0.722 | 1.287 | 0.130 | 1.254 | 0.075 | 0.065 | 0.152 | 0.074 |
| 0.331 | 0.876 | 0.100 | 0.857 | 0.075 | 0.083 | 1.473 | 0.229 |
| 0.107 | 0.150 | 0.058 | 0.135 | 0.049 | 0.046 | 0.344 | 0.146 |
| 0.333 | 0.594 | 0.152 | 0.522 | 0.128 | 0.111 | 0.245 | 0.172 |
| 0.247 | 0.339 | 0.109 | 0.286 | 0.084 | 0.060 | 0.339 | 0.151 |
| Rhip | Hotv | Mona | Akari | Aust | Belil | Felis | LPS |

EIA modules were coated with near optimal antigen dilutions overnight at ambient temperature, back coated with WellChampion® (Kementec, Denmark) and dried overnight. A commercial kit protocol and components (RRM-96K, Fuller Laboratories) were utilized throughout, including a serum pre-treatment step for IgM testing which removes the majority of competing IgG antibody. Test and control wells were quantitated at 450 nm and absorbance values compared with the apparent baseline values to determine the level of reactivity. The order of these antigens in Table 1 (1 through 18) is based on relatedness of OmpB.

Each of the serum test results (A₄₅₀ nm) constitute a row across two ELISA plates, while the specific antigen is listed below the column coated with that antigen. These IgM results demonstrated that the species share a common OmpB epitope that has been blocked in half of the antigen preps by the heteroduplex formation with OmpA.

In contrast, a commercial IgM assay (RRM-96K) uses R. rickettsii antigen in which the IgM binding has not been blocked and validates the high sensitivity and specificity of this assay. The substitution of this IgM-binding epitope of OmpB produces a more economical ELISA, while making it possible to produce equally sensitive and specific rapid test formats possible. This assay will also be converted to this rapid test format wherein the ˜32 kDa binding epitope will be conjugated to a visual marker (i.e. colloidal gold) and utilized to bind captured patient IgM-class antibody on a strip test format.

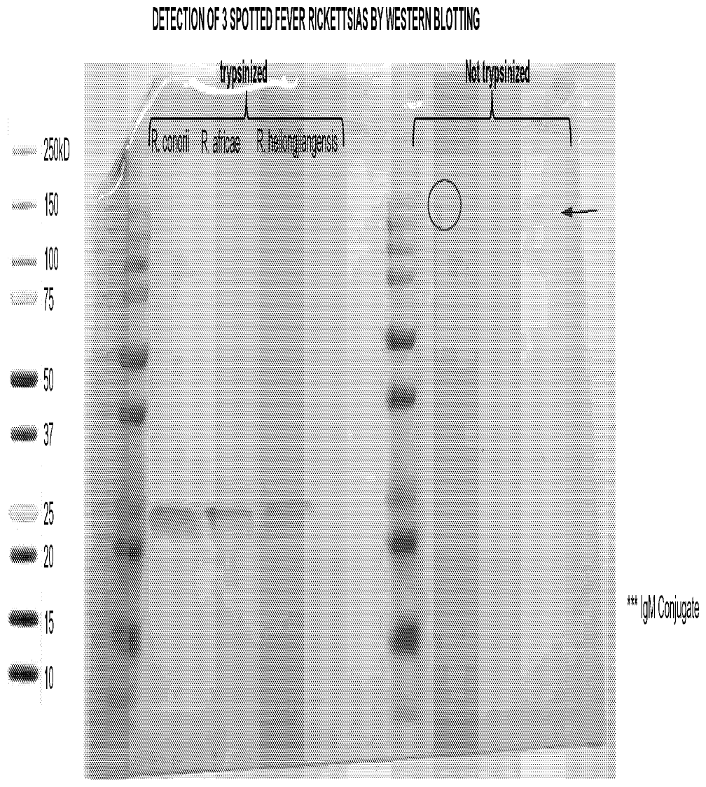

A Western blot was produced using two antigens that had been blocked by OmpA and one (R. conorii) that had not in the above 18-antigen assay. On the left side the blot was treated overnight with trypsin and the ˜25 kDa epitope is seen in all three examples (FIG. 1).

In embodiments, the β-peptide is the antigen to be used in disclosed assays. This β-peptide specifically binds the host immune IgM made in response to recent and/or active infection (or vaccination) by either or both spotted fever and typhus group Rickettsia, but not non-immune IgM or IgG directed against Rickettsia. Immune IgG is, in contrast, directed against the OmpB (and OmpA) passenger domains (and other antigens), not the β-peptide. This β-peptide is conserved within the spotted fever group at a level of approximately 95% and also 75% within the typhus fever group.

### Example 2—Antigen Comparison

An enzyme-immunoassay (EIA) was performed to compare antigens. Antigen (here each diluted antigen had a single-column) was added 100 uL/well overnight, then directly back-coated (add 100 ul for 10 minutes) to preserve antigenicity, then emptied and dried for packaging.

Sera were added to the individual rows at 1:100 in PBS containing Tween-20 (standard EIA wash buffer) and incubated for 60 minutes at ambient temperature (˜23 C). Wells are then washed 3× with this Wash Buffer, tapped to remove buffer before adding 100 uL peroxidase-conjugated anti-human IgG for 30 minutes (same temp). After washing again, the substrate (TMB) is added for a timed 10 minutes and the reaction is stopped by addition of 100 uL of 10% NaOH. An EIA reader set to 450 nm is used to read the absorbance for each well and those are the numbers in the current text (within my computer form).

These absorbance values can simply be compared, but in clinical assays a Cut-Off Serum is provided that is squarely between positive and negative. Higher values than this Cut-Off are positive and lower values denote negative (+/−10% equivocal).

### Example 3—Treatment of Rickettsia

A 30-year old female patient is suspected of having a Rickettsia infection.

Sera from the patient is tested as described herein, and a positive result recorded. The patient is treated for 10 days with doxycycline.

### Example 4—Treatment of Rickettsia

A 20-year old male patient is suspected of having a Rickettsia infection.

Sera from the patient is tested as described herein, and a positive result recorded. The patient is treated for 12 days with chloramphenicol.

### Example 5—Treatment of Rickettsia

A 50-year old female patient is suspected of having a Rickettsia infection.

Sera from the patient is tested as described herein, and a negative result recorded. Further tests are performed to determine the cause of the patient's symptoms.

### Disclosed Embodiments

Embodiment 1. A purified, recombinant, non-naturally occurring, non-post-translationally modified polypeptide having a sequence with at least 70% identity to the sequence as set forth in SEQ ID NO:2.
Embodiment 2. The polypeptide of embodiment 1, wherein the polypeptide has a sequence with at least 80% identity to the sequence as set forth in SEQ ID NO:2.
Embodiment 3. The polypeptide of embodiment 2, wherein the polypeptide has a sequence with at least 90% identity to the sequence as set forth in SEQ ID NO:2.
Embodiment 4. The polypeptide of embodiment 3, wherein the polypeptide has a sequence with at least 95% identity to the sequence as set forth in SEQ ID NO:2.
Embodiment 5. The polypeptide of embodiment 4, wherein the polypeptide has a sequence with at least 98% identity to the sequence as set forth in SEQ ID NO:2.
Embodiment 6. An Enzyme-Linked Immunosorbent Assay (ELISA) kit comprising the polypeptide of embodiments 1-5.
Embodiment 7. The kit of embodiment 6, further comprising a 96-well plate.
Embodiment 8. The kit of embodiment 7, further comprising phosphate-buffered saline (PBS).
Embodiment 9. The kit of embodiment 8, further comprising NaOH.
Embodiment 10. The kit of embodiment 9, further comprising 3,3′,5,5″-tetramethylbenzidine (TMB).
Embodiment 11. A method of detecting exposure to Rickettsia in an animal or in a biological sample from said animal, the method comprising the step of detecting the presence of an immune response to a polypeptide having the amino acid sequence as set forth in SEQ ID NO:2.
Embodiment 12. The method of embodiment 11, wherein said detecting the presence of an immune response comprises detecting antibody binding to the polypeptide having the amino acid sequence as set forth in SEQ ID NO:2.
Embodiment 13. The method of embodiment 12, wherein said exposure to Rickettsia comprises spotted fever or typhus group Rickettsia.
Embodiment 14. A purified, recombinant, non-naturally occurring peptide having a sequence with at least 70% identity to the sequence as set forth in SEQ ID NO:2.
Embodiment 15. A purified, recombinant, non-naturally occurring peptide having a sequence with at least 80% identity to the sequence as set forth in SEQ ID NO:2.
Embodiment 16. A purified, recombinant, non-naturally occurring peptide having a sequence with at least 90% identity to the sequence as set forth in SEQ ID NO:2.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A purified, recombinant, non-naturally occurring, non-post-translationally modified Rickettsia OmpB polypeptide having a sequence with at least 70% identity to the sequence as set forth in SEQ ID NO:2.

2. The polypeptide of claim 1, wherein the polypeptide has having a sequence with at least 80% identity to the sequence as set forth in SEQ ID NO:2.

3. The polypeptide of claim 2, wherein the polypeptide has having a sequence with at least 90% identity to the sequence as set forth in SEQ ID NO:2.

4. The polypeptide of claim 3, wherein the polypeptide has having a sequence with at least 95% identity to the sequence as set forth in SEQ ID NO:2.

5. The polypeptide of claim 4, wherein the polypeptide has having a sequence with at least 98% identity to the sequence as set forth in SEQ ID NO:2.

6. An Enzyme-Linked Immunosorbent Assay (ELISA) kit comprising the polypeptide of claims 1-5.

7. The kit of claim 6, further comprising a 96-well plate.

8. The kit of claim 7, further comprising phosphate-buffered saline (PBS).

9. The kit of claim 8, further comprising NaOH.

10. The kit of claim 9, further comprising 3,3′,5,5″-tetramethylbenzidine (TMB).

11. A method of detecting exposure to Rickettsia in an animal or in a biological sample from said animal, the method comprising the step of detecting the presence of an immune response to the polypeptide having the amino acid sequence as set forth in SEQ ID NO:2.

12. The method of claim 11, wherein said detecting the presence of an immune response comprises detecting antibody binding to the polypeptide having the amino acid sequence as set forth in SEQ ID NO:2.

13. The method of claim 12, wherein said exposure to Rickettsia comprises spotted fever or typhus group Rickettsia.

14. A purified, recombinant, non-naturally occurring peptide having a sequence with at least 70% identity to the sequence as set forth in SEQ ID NO:2.

15. A purified, recombinant, non-naturally occurring peptide having a sequence with at least 80% identity to the sequence as set forth in SEQ ID NO:2.

16. A purified, recombinant, non-naturally occurring peptide having a sequence with at least 90% identity to the sequence as set forth in SEQ ID NO:2.


SEQUENCE LISTING (3 sequences)

SEQ ID NO: 1 - DNA, 1008 bp, synthetic construct
  source 1..1008: mol_type = other DNA
atggcgggcccggaagcgggcgcgattccggcggcggtggcggcgggcgatgaagcggtggataacgtggcgtatggcatttgggcgaaaccgttttataccgatgcgcatcagagcaaaaaaggcggcctggcgggctataaagcgaaaaccaccggcgtggtgattggcctggataccctggcgaacgataacctgatgattggcgcggcgattggcattaccaaaaccgatattaaacatcaggattataaaaaaggcgataaaaccgatgtgaacggctttagctttagcctgtatggcgcgcagcagctggtgaaaaacttttttgcgcagggcagcgcgatttttagcctgaaccaggtgaaaaacaaaagccagcgctatttttttgatgcgaacggcaacatgagcaaacagattgcggcgggccattatgataacatgacctttggcggcaacctgaccgtgggctatgattataacgcgatgcagggcgtgctggtgaccccgatggcgggcctgagctatctgaaaagcagcgatgaaaactataaagaaaccggcaccaccgtggcgaacaaacaggtgaacagcaaatttagcgatcgcaccgatctgattgtgggcgcgaaagtggcgggcagcaccatgaacattaccgatctggcggtgtatccggaagtgcatgcgtttgtggtgcataaagtgaccggccgcctgagcaaaacccagagcgtgctggatggccaggtgaccccgtgcattagccagccggatcgcaccgcgaaaaccagctataacctgggcctgagcgcgagcattcgcagcgatgcgaaaatggaatatggcattggctatgatgcgcagattagcagcaaatataccgcgcatcagggcaccctgaaagtgcgcgtgaactttggcctgaatgatattttcgaagcacagaaaattgaatggcacgaactggtgcctcgtggaagtcaccatcatcaccaccactag

SEQ ID NO: 2 - protein, 308 aa, synthetic construct
MAGPEAGAIPAAVAAGDEAVDNVAYGIWAKPFYTDAHQSKKGGLAGYKAKTTGVVIGLDTLANDNLMIGAAIGITKTDIKHQDYKKGDKTDVNGFSFSLYGAQQLVKNFFAQGSAIFSLNQVKNKSQRYFFDANGNMSKQIAAGHYDNMTFGGNLTVGYDYNAMQGVLVTPMAGLSYLKSSDENYKETGTTVANKQVNSKFSDRTDLIVGAKVAGSTMNITDLAVYPEVHAFVVHKVTGRLSKTQSVLDGQVTPCISQPDRTAKTSYNLGLSASIRSDAKMEYGIGYDAQISSKYTAHQGTLKVRVNF

SEQ ID NO: 3 - protein, 348 aa, synthetic construct
AGPEAGAIPAAVAAGDEAVDNVAYGIWAKPFYTDAHQSKKGGLAGYKAKTTGVVIGLDTLANDNLMETIGAAIGITKTDIKHQDYKKGDKTDVNGFSFSLYGAQQLVKNFFAQGSAIFSLNQVKNKSQRYFFDANGNMETSKQIAAGHYDNMETTFGGNLTVGYDYNAMETQGVLVTPMETAGLSYLKSSDENYKETGTTVANKQVNSKFSDRTDLIVGAKVAGSTMETNITDLAVYPEVHAFVVHKVTGRLSKTQSVLDGQVTPCISQPDRTAKTSYNLGLSASIRSDAKMETEYGIGYDAQISSKYTAHQGTLKVRVNFGLNDIFEAQKIEWHELVPRGSHHHHHH